(19) United States
(12) Patent Application Publication
(10) Pub. No.: US 2014/0083420 A1
(43) Pub. Date: Mar. 27, 2014

(54) INHALATION ANESTHETIC VAPORIZER

(71) Applicant: Piramal Critical Care, Inc., Orchard Park, NY (US)

(72) Inventors: Ritchie Robel, Hamburg, NY (US); William Burns, Orchard Park, NY (US); Chris Noriji, Victoria, TX (US); Michael Gwaze, Buffalo, NY (US); Michael Cuzydlo, Orchard Park, NY (US)

(73) Assignee: Piramal Critical Care, Inc., Orchark Park, NY (US)

(21) Appl. No.: 14/037,921

(22) Filed: Sep. 26, 2013

Related U.S. Application Data
(60) Provisional application No. 61/705,965, filed on Sep. 26, 2012.
(60) Provisional application No. 61/801,209, filed on Mar. 15, 2013.

(51) Int. Cl. A61M 16/18

(52) U.S. Cl. CPC A61M 16/18
USPC 128/203.14

(57) ABSTRACT

A vaporizer includes a thermally conductive anesthetic container and a temperature sensor positioned in the container and in the liquid anesthetic. A breathing-gas delivery tube has a delivery end positioned in the liquid anesthetic at least about 5 mm beneath the top surface of the liquid anesthetic. A heater is positioned outside the container. Control circuitry is configured to receive signals from the temperature sensor and is connected to the heater in order to cause the heater to maintain the temperature of the anesthetic above a predetermined temperature. A method of vaporizing inhalation anesthetic is also provided.

### RELATED APPLICATION

This application claims priority from U.S. Provisional Applications Nos. 61/705,965, filed 26 Sep. 2012, and 61/801,209, filed 15 Mar. 2013, the subject matter of both of which is incorporated herein by reference in its entirety.

### TECHNICAL FIELD

The present invention relates to an apparatus and method for use of an inhalation anesthetic vaporizer.

### BACKGROUND OF THE INVENTION

Inhalation anesthetics are commonly delivered to patients by a vaporizer.

The scientific principles that relate the operation of a vaporizer include vapor pressure, boiling point of volatile liquid anesthetic agents, flow rate, gas concentration, heat of vaporization, specific heat, and thermal conductivity. An anesthetic vaporizer design may take into account these principles.

It is known in the art that the vapor pressure of an anesthetic gas is affected by variables such as temperature and pressure. In the context of volatile liquid anesthetics, vapor pressure is the pressure exerted by a vapor in thermodynamic equilibrium with its liquid phase at a given temperature in a closed system. The vapor pressure of a given volatile liquid anesthetic increases non-linearly with temperature in a closed system. The vapor pressure that a single component in a mixture (e.g., sevoflurane component in a mixture of sevoflurane agent vapor and carrier gas) contributes to the total pressure in the system is called partial pressure. It is also known in the art that, as the anesthetic in the vaporizer evaporates, the vaporizer chamber will cool (more fully explained below), thus affecting the vapor pressure of the anesthetic agent and the amount of anesthetic agent that is entrained by the carrier gas. To maintain a proper amount of anesthetic agent, the flow rate of the carrier gas is typically adjusted at the inlet of the carrier gases to the anesthetic machine, and such adjustments are made manually.

Generally speaking, inhalation liquid anesthetic agents are volatile, and require varying degrees of thermal excitation to be transformed from liquid to vapor, which is determined by the specific heat of the anesthetic. When the temperature of the anesthetic inside a closed container is raised, molecules of the liquid will break away from the surface and enter the surface above it, forming vapor. These molecules bombard the walls, creating a pressure called the vapor pressure. If the container is kept at a set temperature, eventually equilibrium is formed between the liquid and vapor phases so that the number of molecules in the vapor phase remains constant. If, after equilibrium, heat is supplied to the container, the equilibrium will be shifted so that more molecules will enter the vapor phase, creating a higher vapor pressure. On the other hand, if heat is taken away, molecules will return to the liquid phase and the vapor pressure will be lower. This ability to increase or lower the vapor pressure affects the concentration of the anesthetic vapor and the accuracy of the vaporizer output regardless of the dial setting. Vapor pressure of an anesthetic agent changes with variables such as temperature and pressure. Since the vapor pressure of an anesthetic changes with temperature, the dialed concentration will not be equal to the delivered concentration. It is known that energy is required for the molecules of a liquid to change into vapor. This is supplied by the liquid itself. Regardless of the vaporizer setting, without thermal compensation, the vaporizer cools as the liquid anesthetic vaporizes, and results in a decrease in concentration, or volume percent delivered to the patient. This heat of vaporization is supplied by the remaining anesthetic (temperature loss by the latent heat of vaporization) causing further drop in temperature of a traditional vaporizer chamber and decrease in vaporizer output over time. This drop in temperature in a traditional vaporizer chamber is due to evaporative cooling, caused by the evaporation of the anesthetic agent as noted above. In other words, the amount of anesthetic agent entrained will decrease over time. To counter this phenomenon and to assure accuracy of anesthetic delivery, known anesthetic vaporizers may provide for temperature compensation means with the temperature of the vaporizer chamber maintained at a set temperature of between 25-35° C.

Vaporizer performance and, thus, quality of an anesthesia machine are affected by how well the vaporizer is designed to insulate it from the effects of temperature, pressure, and carrier gas flow rates. A basic design standard prior art vaporizer (conventional bypass type vaporizers) used for delivering liquid anesthetic agents (e.g., sevoflurane, isoflurane, and/or enflurane) is developed around three factors:

Fresh gas (breathing-gas, e.g., oxygen, nitrous oxide, and/or air) flows to a manifold. Here, a splitting valve divides the fresh gas into two flows or fractions: bypass flow and vaporizer chamber flow. The bypass fraction is fresh gas and the vaporizer fraction (carrier gas) flows to the vaporizer and picks up pure anesthetic agent as anesthetic agent forms a saturated vapor and as the carrier gas is passed over sump filled with liquid anesthetic agent. The carrier gas with the anesthetic agent entrained therein exit the vaporizer. These two flows then mix together (forming total flow) before delivery to the patient. The agent concentration is determined by the ratio of gas flow that goes through the vaporizing chamber and the fresh gas that flow through the bypass. The percentage of anesthetic gas fraction that is delivered from the vaporizer is equal to the percent concentration of the anesthetic in the total flow.

Inside the vaporizer chamber, wicking may be used to increase the contact surface area to increase the amount of pure agent vapor inside the chamber. This increased wicking increases the evaporation, which decreases the temperature of the chamber and agent vaporization.

To aid the decreased agent vaporization problem, the addition of a resistance to flow entering the vaporizing chamber may be present, with more resistance to chamber flow at high agent temperature and less resistance to chamber flow at low agent temperature.

The anesthetic sump is maintained to a temperature of between 25-35° C. based upon the specific anesthetic agent and/or provided with wicks inside the vaporizer chamber so as to create a constant production of agent vapor within the sump. However, during high-flow and/or long duration usage, both the temperature compensation for flow and vaporizer mass cannot control the energy loss in the system, which will decrease the vaporizer concentration in the chamber and reducing agent concentration from the vaporizer. Over time, the amount of agent concentration will decrease (see Tec3, FIG. 4) and the vaporizer manufacturers often include a flow characteristic graph in the operation and maintenance manual to help a user compensate for this decrease. Vaporizer output of prior art bypass type vaporizers is dependent on the rate of carrier gas fraction flowing into the vaporizer and does not remain constant at low temperatures in the range of 18° C. to 23° C., for at least the reasons noted above. Moreover, these prior art conventional bypass type vaporizers are not suitable for use with desflurane, for reasons known to those of ordinary skill in the art.

Also, certain currently available commercial vaporizers by design are agent-specific and utilize index systems or other coupling designs to prevent accidental filling with the wrong agent. This means that healthcare providers must purchase multiple vaporizers (one per agent), which may be cost-prohibitive in an era of cost containments and reimbursement cuts. In addition, currently available commercial vaporizers are bulky, heavy, and/or complex to set up and maintain, and require tremendous logistical overhead to transport. The challenges posed by currently available vaporizers and their performance make it desirable to provide a simple and cost-effective vaporizer with basic features that can achieve consistent precision and reliability not found in any commercially available anesthetic vaporizer.

### SUMMARY OF THE INVENTION

In an embodiment of the present invention, a vaporizer is described. The vaporizer includes a thermally conductive anesthetic container and a temperature sensor positioned in the container and in the liquid anesthetic. A breathing-gas delivery tube has a delivery end positioned in the liquid anesthetic at least about 5 mm beneath the top surface of the liquid anesthetic. A heater is positioned outside the container. Control circuitry is configured to receive signals from the temperature sensor and is connected to the heater in order to cause the heater to maintain the temperature of the anesthetic above a predetermined temperature.

In an embodiment of the present invention, a method of vaporizing inhalation anesthetic is described. A vaporizer is provided, the vaporizer having a thermally conductive anesthetic container and a temperature sensor positioned in the container and in the liquid anesthetic. A breathing-gas delivery tube has a delivery end positioned in the liquid anesthetic. A heater is positioned outside the container. Control circuitry is configured to receive signals from the temperature sensor and is connected to the heater to cause the heater to maintain the temperature of the anesthetic above a predetermined temperature. The container is at least partially filled with liquid anesthetic. Breathing-gas is provided to the delivery tube to cause breathing-gas to bubble through the liquid anesthetic. The temperature of the liquid anesthetic is determined using the sensor and the control circuitry. When the liquid anesthetic temperature is below a set point, the heater provides heat to the container until the temperature achieves the set point.

In an embodiment of the present invention, a vaporizer is described. A thermally conductive anesthetic container contains liquid anesthetic. A temperature sensor is positioned in the container and in the liquid anesthetic. A breathing-gas delivery tube has a delivery end positioned in the liquid anesthetic beneath the top surface of the liquid anesthetic. A heater is positioned outside the container and configured to supply heat to the liquid anesthetic via conduction through the anesthetic container. Control means are configured to receive signals from the temperature sensor and responsively control the heater to maintain the temperature of the anesthetic at least one of at and above a predetermined temperature.

### BRIEF DESCRIPTION OF THE DRAWINGS

For a better understanding of the invention, reference may be made to the accompanying drawings, in which:

FIG. 1 is a schematic view of a vaporizer of one embodiment of the present invention;

FIG. 2 is a partial schematic view of a vaporizer of the embodiment of FIG. 1;

FIG. 3 is a partial schematic view of a vaporizer of the embodiment of FIG. 1;

FIG. 4 is a graph depicting a comparison between a vaporizer of the embodiment of FIG. 1 (“WV”) and a prior art vaporizer (“TEC 3”);

FIG. 5 is a graph showing a comparison between a vaporizer of the embodiment of FIG. 1 (“WV”) and prior art vaporizers (“Draeger D-Vapor 2000”, “Penlon Sigma Delta”, and “GE Tec 7”);

FIG. 6 is a temperature graph;

FIG. 7 shows a concentration curve display on an Ohmeda 5330 agent monitor;

FIG. 8 shows a temperature graph for varying flow rates; and

FIG. 9 shows concentration graph display for varying flow rates on an Ohmeda 5330 agent monitor.

### DESCRIPTION OF EMBODIMENTS

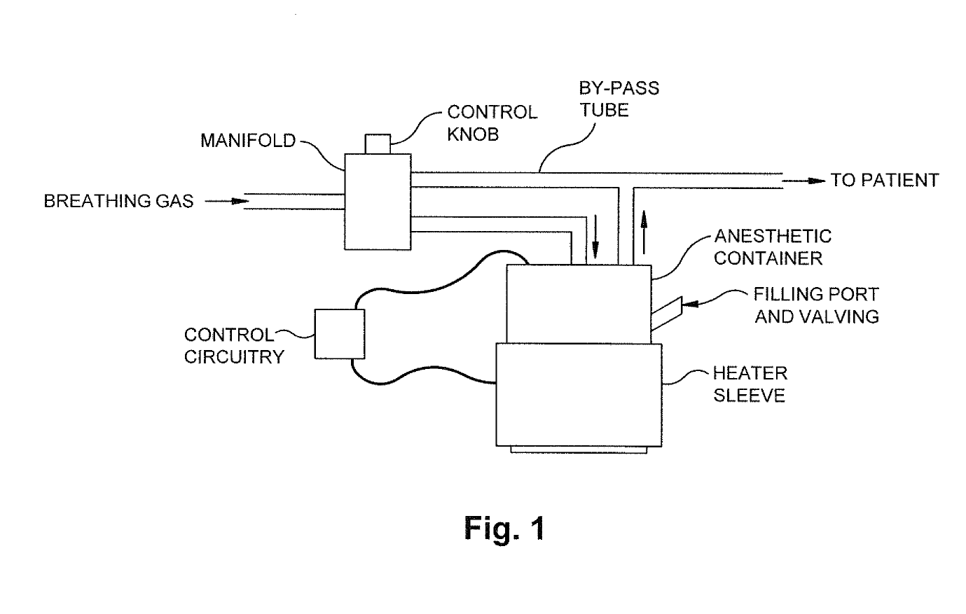

In accordance with the present invention, FIG. 1 depicts an anesthetic vaporizer. As described herein, the present invention addresses the challenges posed by prior art vaporizers, in that (i) latent heat of vaporization results in a drop in drug temperature that may result in a lower partial pressure, and (ii) lower partial pressure may result in lower concentration. The temperature of a given anesthetic for the device of FIG. 1 is a maintained at a set temperature ±0.5° C. or 1.0° C. (e.g., at 22° C.±0.5° C. or 22° C.±1.0° C.).

The vaporizer shown and described herein may incorporate at least the following aspects:

the vaporizer inflow tube is placed below the surface of the liquid anesthetic agent such that the outlet of the vaporizer inflow tube (or the delivery end of the delivery tube) is located anywhere from at least about 5 mm from the surface and no closer than about 2 mm (the term “about” in either of these instances being ±1 mm) to the bottom (relative to gravity) of the container, but not touching the bottom, to allow the carrier gas bubbling the anesthetic liquid agent;

the heating jacket allows the temperature of the vaporizer chamber (sump) with liquid agent to remain at the set temperature (which can be, for example, below the desflurane boiling point of 22.8° C. at 757 mm Hg) (e.g., at 22° C.), to ensure that the same amount of saturated agent is in the chamber.

The described vaporizer can keep the pressure in the chamber substantially constant, such that the partial pressure of the anesthetic agent will remain substantially constant, and hence, the percent of anesthetic agent entrained will remain substantially constant. Vaporizer output may remain substantially constant at a set temperature which can be any of 18° C. to 29° C.±1° C., independent of the rate of carrier gas fraction flowing into the vaporizer.

The described vaporizer may be used for delivery of anesthetic agents such as, but not limited to, sevoflurane, isoflurane, desflurane, and/or enflurane. As described herein, the vaporizer can include at least:

A splitting valve which divides the fresh gas into two flows: Bypass flow and Vaporizer (chamber) flow. The Bypass is fresh gas, and Vaporizer Flow is gas that mixes pure agent vapor. These two flows then mix together in the delivery to the patient. The percent Anesthetic Gas Concentration equals the anesthetic gas fraction (fA) plus the carrier gas fraction (fC) flowing out of the vaporizer, divided by total gas outflow (fT) multiplied by 100 (this relationship can also be written as % Anesthetic Gas Concentration (fA+fC)/fT×100). An example of a suitable maximum total gas outflow (fT) may be, for example, 15 LPM (liters per minute).

Inside the vaporizer chamber, the Vaporizer inflow tube from the splitting is placed below the surface of the liquid anesthetic agent at a depth previously discussed. The carrier gas is bubbled up through the liquid anesthetic agent to produced saturated agent vapor.

On the outside of the vaporization chamber, a heating jacket may be used to help keep the agent at a controlled temperature. Temperature sensors and control circuitry may be provided for monitoring the temperature of the liquid anesthetic agent and vapor agent inside the chamber and for controlling the amount of heat provided to the chamber by the heating jacket. The heating jacket may be positioned to apply heat to the sides of the chamber, the bottom of the chamber, and/or the top of the chamber, as desired for a particular use environment of the present invention.

This vaporizer design removes the prior art conventions of wicking and the use of large amounts of anesthetic agent mass and compensation for vaporizer inflow to aid in the energy loss during normal uses. At least by controlling temperature variables in the design and by bubbling, the vaporizer described herein is able to deliver the same agent concentration as prior art vaporizers, independent of the flow rate of the carrier gas flowing into the vaporizer chamber. Unlike in the prior art vaporizers, the rate of carrier gas flow into the sump or length of time of operation does not change the concentration delivered, even when the temperature of the agent in the sump is maintained at temperatures below the desflurane boiling point.

The operation of this vaporizer is, for example, as follows:

The temperature of the liquid agent is controlled, such as, for example, to a maximum temperature below the desflurane boiling point of 22.8° C. at 757 mmHg) (e.g., a maximum temperature of 22° C.) for desflurane and other anesthetic agents or, as another example, far below the boiling point of sevoflurane. A temperature sensor is suspended in the agent near, but not touching, the bottom of the chamber. A second sensor may be used to monitor the temperature of the vapor agent. The maximum permissible temperature measured via either of these sensors will generally be below the boiling point of the agent in the vaporizer chamber, and the temperature of the chamber will be controlled as discussed below to avoid the chamber reaching that maximum permissible temperature.

The vaporizer inflow tube outlet is placed anywhere from at least about 5 mm from the surface and no closer than about 2 mm to the bottom, but not touching the bottom. It is believed that the bubbling of the carrier gas from the inflow tube into the agent aids in efficiency of the process of producing saturated agent vapor in the chamber.

Reduction of the liquid agent temperature from evaporation of the agent liquid (or heat loss by the latent heat of vaporization) is prevented by the heating jacket keeping the liquid at the set temperature (e.g., 22° C.). If the liquid agent temperature is greater than the controlling temperature, evaporation will cool the agent down to the controlling set point.

The vaporizer outflow tube is designed for a back pressure to keep the output concentration the same for different fresh gas flow.

FIG. 1 is a schematic representation of an anesthetic vaporizer according to an embodiment of the present invention. Anesthetics such as, but not limited to, sevoflurane, desflurane, isoflurane, and/or enflurane may be delivered to a patient with this vaporizer. Such a vaporizer may include an anesthetic container, a heater, and a breathing-gas delivery tube. A manifold receives breathing-gas from a source and directs some of the breathing-gas to the interior of the container where it is bubbled through the liquid anesthetic. The manifold also directs some of the breathing-gas to a tube that bypasses the container. Anesthetic vapor from the container is mixed with the bypassed breathing-gas, and this mixture is delivered to the patient. A control knob may be provided to allow adjustment of the amount of breathing-gas delivered to the container, and thereby the amount of anesthetic delivered to the patient.

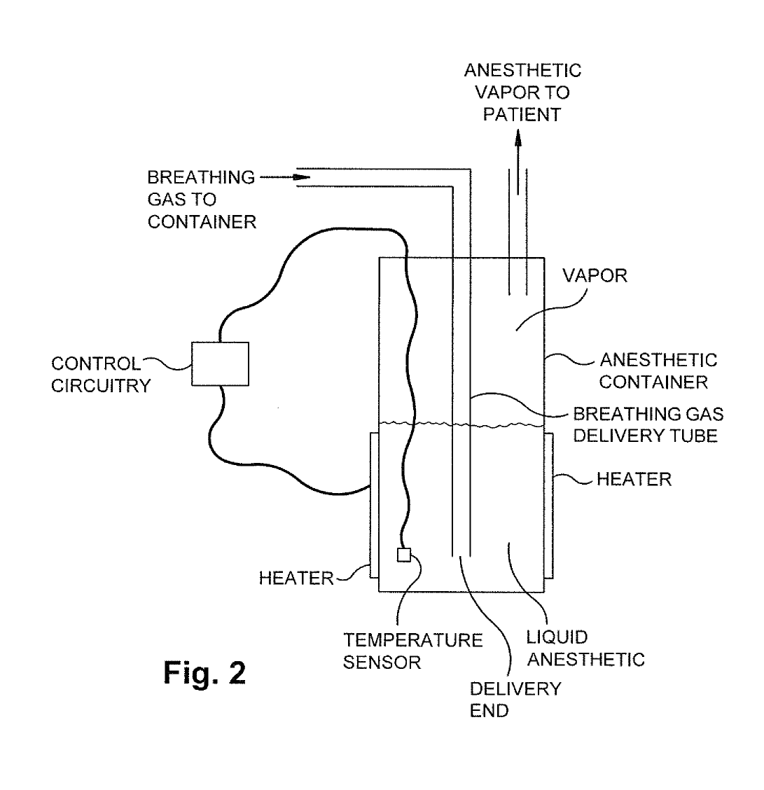

FIG. 2 is a schematic representation of the vaporizer which depicts additional details of the embodiment of FIG. 1. The invention may include a temperature sensor and control circuitry for monitoring the temperature of the liquid anesthetic and/or for controlling the amount of heat provided to the container by the heater. As liquid anesthetic vaporizes, heat energy is provided to the liquid anesthetic so as to maintain the temperature of the liquid anesthetic, and thereby offset the energy associated with vaporizing the liquid.

The anesthetic container may be made of one or more highly thermally conductive materials. For example, the container may be made from copper, aluminum, gold, or silver. The container may also or instead be made from any other suitable thermally conductive materials. The container may also be made from a material which does not react to the anesthetic contained within when heat is applied to the container.

When heat is applied to the exterior of the container by the heater, the container conducts the heat to the interior of the container, and ultimately to the liquid anesthetic. The container may be substantially cylindrical in order to (among other reasons) more evenly deliver heat to the liquid anesthetic. The container may also include a flange. The heater may be configured to apply heat, sequentially or simultaneously, to the sides, top, and/or bottom of the container. It should be noted that the bottom of the container referred to herein describes the bottom relative to gravity.

Furthermore, heat might be applied only to the portions of the exterior container sides which have a corresponding interior side that is at least partially in contact with the liquid anesthetic. In this manner, the heat conduction path from the heater to the liquid may be minimized. To achieve such an arrangement, a heater sleeve may be positioned around the lower portion of the sides of the container. Such a heater sleeve may provide heat using electric resistance derived from electricity regulated by the control circuitry. It is believed that a sleeve operating at about 115 Volts and in the range of about 30-70 Watts (e.g., about 67.5 Watts) may suffice. Furthermore, the sleeve may be configured to fit closely to the exterior surface of the container.

The temperature sensor may include an electronic sensor positioned in the liquid anesthetic. For example, the temperature sensor may be a thermistor positioned near the bottom of the container. Preferably, the temperature sensor does not contact the container or is thermally insulated from the container so that the information produced by the temperature sensor is highly indicative of the liquid temperature. In one example embodiment of the invention, the temperature sensor may be located at least 2 mm from the bottom of a cylindrical container, and at least 15 mm from the nearest container side.

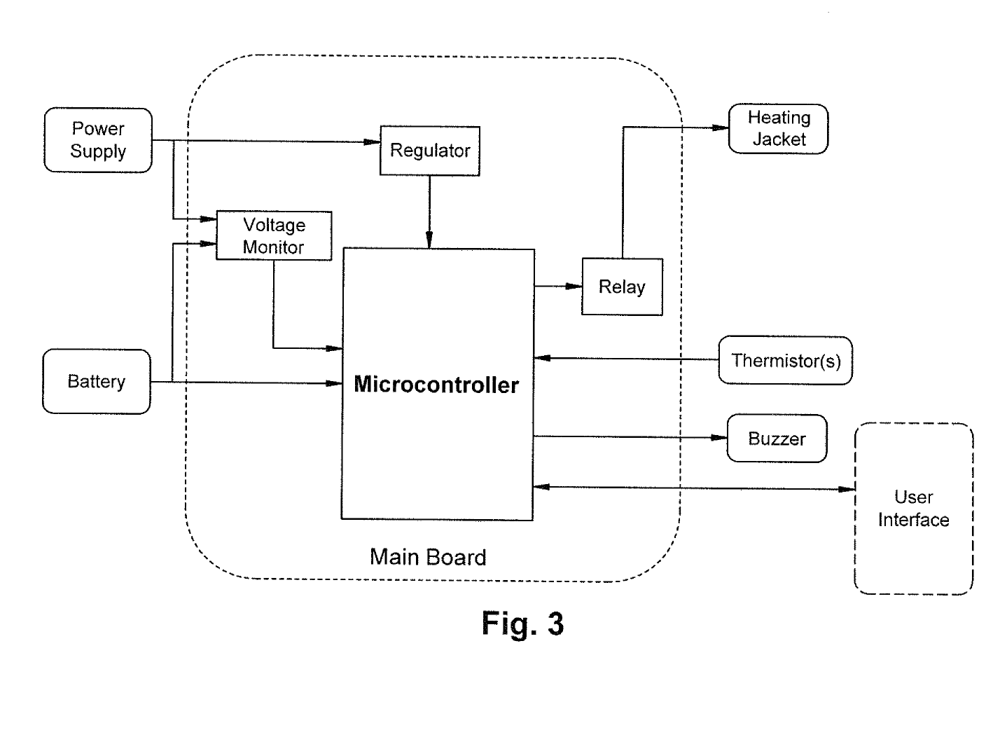

FIG. 3 is a schematic view of an example arrangement of the temperature sensing and control electronics of the present invention. Information signals from the temperature sensor may be provided to the control circuitry, which compares the sensor signals to a set point. If the temperature indicated by the sensor is below the set point, the control circuitry may cause the heater to provide thermal energy to the container in order to cause the liquid temperature to rise. If the temperature indicated by the sensor is at or above the set point, then no thermal energy would be provided by the heater. In this manner, the sensor, control circuitry, and heater function together in the embodiment of FIG. 1 (in contrast to the bimetallic strip arrangement used in the prior art) to maintain the temperature of the liquid anesthetic at or above a set point. In some embodiments of the invention, the set point may be about 23° C. or 23.5° C., but set points as low as 18° C. to 20° C. may be desired and are achievable.

It is noteworthy that no wick is necessary to vaporize the liquid anesthetic in the vaporizer of FIG. 1. To facilitate vaporization, breathing-gas is bubbled through the liquid anesthetic. Breathing-gas is delivered to the lower portion of the liquid anesthetic pool. For example, the delivery end of the delivery tube may be positioned below the surface of the liquid anesthetic agent such that the delivery end of the delivery tube is spaced (e.g., submerged by at least about 5 mm) from the surface of the liquid anesthetic agent and/or spaced (e.g., no closer than about 3 mm) from the bottom of the anesthetic container. In one embodiment of the invention, breathing-gas may be delivered at a location that is substantially equidistant from the container sides, but the position of the delivery end of the delivery tube need not be centrally located relative to the container sides.

In addition to facilitating vaporization of the liquid anesthetic, bubbling the breathing-gas through the liquid anesthetic may have the effect of causing the liquid anesthetic to move within the container, and thereby facilitate the transfer of heat to and throughout the liquid anesthetic. The temperature sensor may be located relative to the delivery end of the delivery tube in order to minimize the influence of the breathing-gas temperature upon the temperature sensor

It is contemplated that placing the temperature sensor and the delivery end at least 30 mm apart may help the temperature sensor to more accurately indicate the overall (steady state) temperature of the liquid anesthetic.

The delivery manifold may include a control knob, which may be used by an operator to adjust the amount of anesthetic provided to the patient. For example, the control knob may include a rotary dial that can be turned by the operator in order to select a desired amount of anesthetic to be delivered to the patient. Once set to a desired position, the dial may be locked in the desired position. For example, the dial may be locked by pressing the dial toward a base until the dial becomes engaged with a latch. The latch holds the dial in the depressed position, and in that position the dial is prevented from turning. By pressing the dial again, the latch may be released and the dial thus permitted to rise (via spring bias) to an elevated position. In the elevated position, the operator may turn the dial in order to alter a desired flow of anesthetic to the patient.

In one embodiment of the invention, the flow of breathing-gas to the container may be stopped when the dial is in the elevated position. Such an arrangement allows an operator to quickly stop the flow of anesthetic merely by pressing on the dial to release the latch and allow the dial to rise to the elevated position. When the dial is in the elevated position, breathing-gas alone (no anesthetic) may be delivered to the patient via the bypass tube.

The dial may be configured to position passageways within the delivery manifold to allow, control, and/or restrict, as desired, the flow of breathing-gas to the container. The passageways may be shaped so that rotation of the dial through an arc of a particular size will change the flow rate of the breathing-gas by a certain amount, regardless of whether the change is with respect to a low flow rate or a high flow rate. That is, the delivery manifold provides a range of flow rates, and if the initial flow rate is low, a rotation of the dial by, for example, X degrees (e.g., 3 degrees) will produce a volumetric change in the flow of breathing-gas that corresponds to the change in flow rate that would occur for an X-degree (e.g., 3-degree) rotation when the flow rate is initially high. A manifold providing such a linear relationship between dial rotation and flow rate change, regardless of the initial flow rate, is believed to have beneficial attributes, including: (a) reducing or eliminating a need to calibrate each vaporizer, (b) reducing the time required to produce each vaporizer, and/or (c) making the vaporizer easier to use.

The manifold may be made from a number of materials. Presently, it is believed that high-density polyethylene (“HDPE”) can be used for many of the manifold components. By using HDPE or another, similarly suitable material, the manifold may be configured to be relatively lightweight and low-cost. Such features may be particularly important if the vaporizer is to be provided to developing locations of the world.

A vaporizer according to an embodiment of the present invention may include an indicator for signaling when the vaporizer is outside of the recommended operating conditions (e.g., accuracy limits). For example, an indicator may signal when the anesthetic level is too low. The container may be provided with valving or other structures to allow quick filling of the vaporizer. When the container is being filled, the heater may be controlled to deliver heat to the container at a rate that is substantially higher than under other operating conditions, and so the heater should be sized accordingly. If the heater is appropriately sized, the vaporizer may be used to provide anesthetic to the patient even during filling of the vaporizer with liquid anesthetic.

It is believed that a vaporizer according to an embodiment of the present invention may be configured for operation to maintain, for example, an 8% concentration of a particular type of anesthetic (e.g., sevoflurane) at 10 LPM (liters per minute). Such a vaporizer may have less than half the number of parts normally required for a prior art anesthetic vaporizer, while also being lightweight, small, and inexpensive when compared to currently commercially available vaporizers.

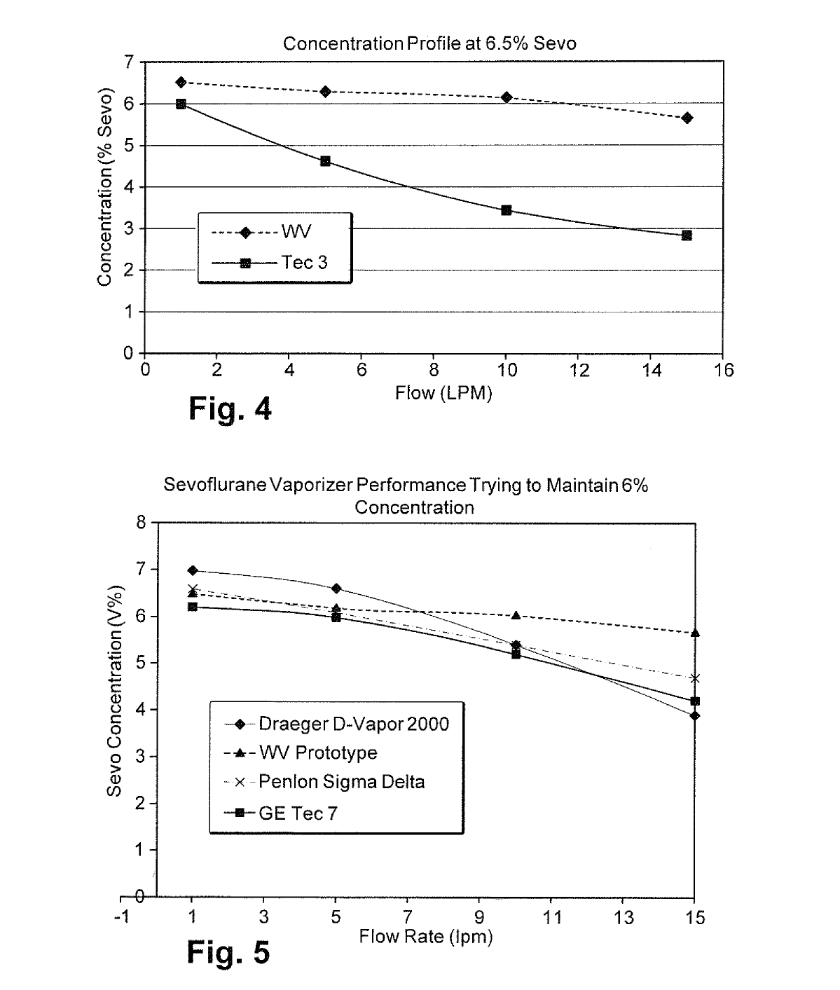

The following examples provide various details including total gas flow rate, anesthetic gas flow rate, percent (%) anesthetic gas concentration, effect of temperature on anesthetic gas concentration, and anesthetic gas concentration concentration stability across different flow rates. In the examples documented in FIGS. 4-9, examples of suitable components forming the anesthetic vaporizer (e.g., WV) include a chamber made using an 8-ounce aluminum bottle, a thermistor, transistor TIP120 Resistors (1 kΩ and 10 kΩ), relay YH1858 (120V, 15 A), breadboard, DC-regulated power supply, Ohmeda 5330 agent monitor, Omega temperature data logger (HH309A), Alicat flow meters, Arduino Duemilanove, and a 115V, 67.5 W heating sleeve. An example anesthetic vaporizer (WV), such as that schematically shown in FIG. 1, was built using these materials.

An aluminum bottle “tank” was used as the anesthetic container—note that a copper tank or other known highly heat-conductive materials may also or instead be used in place of aluminum tank as the anesthetic container. Sevoflurane was the anesthetic used with the container. A silicone rubber heating sleeve (68 W/in²) was used a heating sleeve. Inlet breathing-gas or carrier gas was bubbled into liquid sevoflurane. A thermistor was placed directly into the liquid sevoflurane. Temperature inside the container/chamber tank was controlled using a circuit (such as, but not limited to, the electronics schematically shown at least partially in FIG. 3 and described herein as control means) to control the heating sleeve and thus maintain the temperature inside the tank at a desired level.

The graph in FIG. 4 shows agent concentration drop over time with the known prior art Tec 3 anesthetic vaporizer, but not with the WV, which is a vaporizer embodiment of the present invention, as shown in FIG. 1. (LPM denotes liters per minute in the Figures.)

The graph in FIG. 5 shows agent concentration drop over time with three prior art anesthetic vaporizers (Draeger D-Vapor 2000, Penton Sigma Delta, and GE Tec 7), but not with the WV, which is a vaporizer embodiment of the present invention, as shown in FIG. 1.

A first run (Test 0) was conducted to show that concentration of the agent delivered remains stable over time at 15 LPM of the carrier gas flow into the tank.

TABLE 1

| | | |
|---|---|---|
| Temperature variation | 21-22 | ° C. |
| Concentration variation | 6.6-6.7 | % Sevo |
| Flow | 15 | LPM |
| Bypass | 6.48 | LPM |
| Tank | 9.76 | LPM |

Split Ratio = 0.66

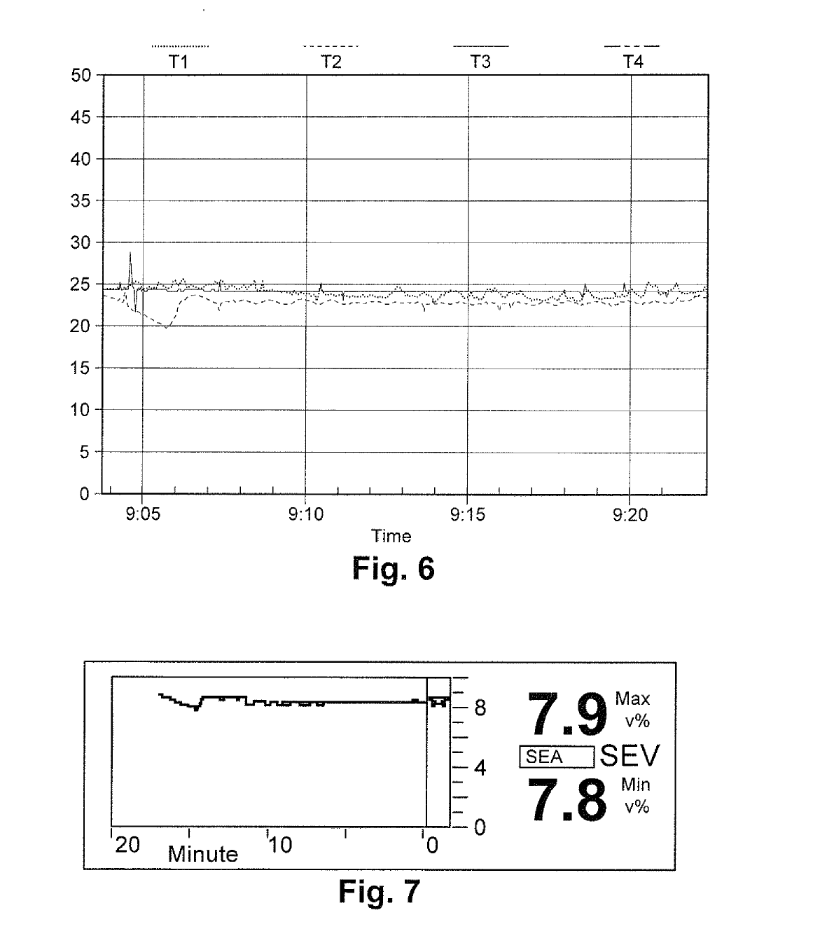

Shown in FIG. 6 is (Test 0, WV) a temperature graph (sevoflurane temperature is labeled as T2). The decline in tank temperature seen in the graph of FIG. 6 is a result of the lack of maintaining temperature (intentionally, for the sake of experiment). Once the tank temperature is maintained, as described below, the concentration then reached a steady state.

Shown in FIG. 7 is (Test 0, WV) concentration curve display on an Ohmeda 5330 agent monitor.

A second run (Test 1) to show stability across different flow rates at one concentration was performed, giving the data in Tables 2-4:

TABLE 2
Results at 1 LPM

| | | |
|---|---|---|
| Temperature variation | 22-23 | ° C. |
| Concentration variation | 6.5-6.6 | % Sevo |
| Total Flow (Into Vaporizer) | 1 | LPM |
| Bypass flow | 0.6 | LPM |
| Tank Flow | 0.4 | LPM |

Split Ratio 1.5

Fᵢₙ=1LPM

F₁=0.6LPM

F₂=0.4LPM

TABLE 3
Results at 5 LPM

| | | |
|---|---|---|
| Temperarture variation | 22-23 | ° C. |
| Concentration variation | 6.3 | % Sevo |
| Total Flow (Into Vaporizer) | 5 | LPM |
| Bypass flow | 3.23 | LPM |
| Tank flow | 1.99 | LPM |

Split Ratio = 1.6

TABLE 4
Results at 10 LPM

| | | |
|---|---|---|
| Temperature variation | 22-23 | ° C. |
| Concentration variation | 6.1-6.2 | % Sevo |
| Total Flow (Into Vaporizer) | 9.67 | LPM |
| Bypass flow | 6.17 | LPM |
| Tank Flow | 4.02 | LPM |

Split Ratio = 1.5

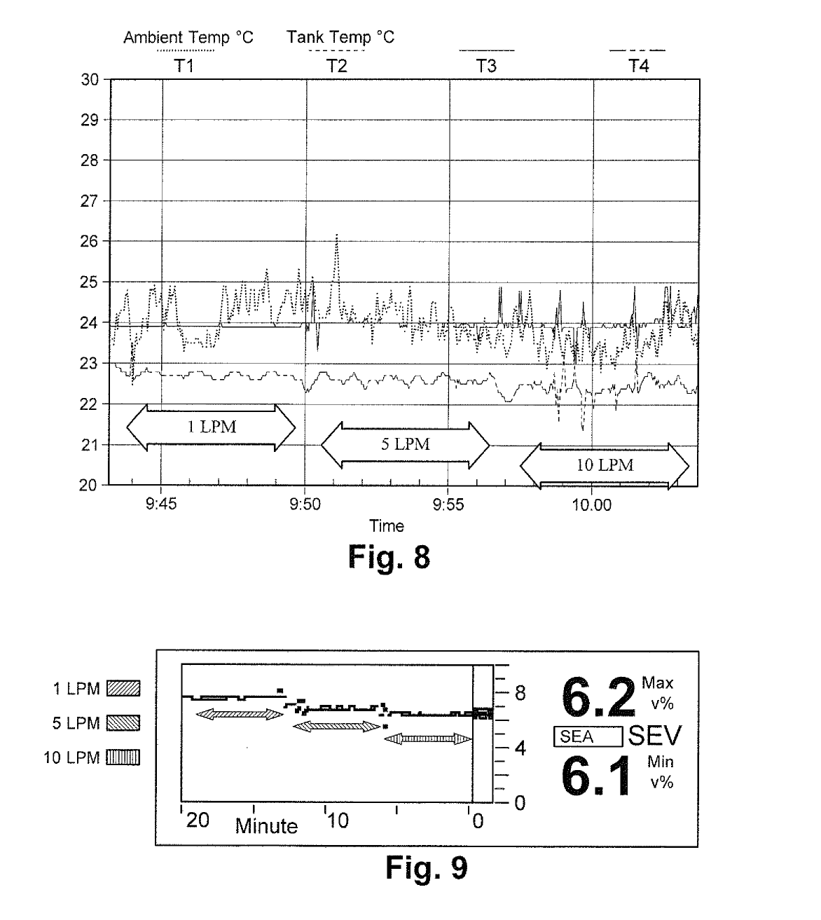

Shown in FIG. 8 is (Test 1, WV) temperature graph for varying flow rates.

Shown in FIG. 9 is (Test 1, WV) concentration graph display, for varying flow rates, on an Ohmeda 5330 agent monitor. See also FIG. 4, showing agent concentration drop over time with Tec 3 but not with the WV, which is a vaporizer embodiment of the present invention, as shown in FIG. 1.

Anesthetic gas (sevoflurane) concentration profile at different temperatures across the testing runs are given in the below Tables 5 and 6.

TABLE 5
Concentration profile when the anesthetic was maintained at 22° C.:
Concentration profile when the anesthetic was maintained at 22° C.

| | | | | | |
|---|---|---|---|---|---|
| | | | | Ratio | Actual |
| Total Input | Actual Input | Bypass | Tank | (Tank/Bypass) | Conc |

Concentration 2%

| | | | | | |
|---|---|---|---|---|---|
| 1 | 1.04 | 0.64 | 0.51 | 0.80 | 2.2 |
| 5 | 5.01 | 4.35 | 0.91 | 0.21 | 2.2 |
| 10 | 10.03 | 9.06 | 1.46 | 0.16 | 2.2 |
| 15 | 15 | 13.81 | 2.02 | 0.15 | 2.2 |

Concentration 4%

| | | | | | |
|---|---|---|---|---|---|
| 1 | 0.97 | 0.68 | 0.4 | 0.59 | 4 |
| 5 | 4.99 | 3.97 | 1.26 | 0.32 | 3.9 |
| 10 | 10 | 8.03 | 2.5 | 0.31 | 4.1 |
| 15 | 15.06 | 12.17 | 3.8 | 0.31 | 4 |

Concentration 6%

| | | | | | |
|---|---|---|---|---|---|
| 1 | 0.97 | 0.51 | 0.57 | 1.12 | 6.1 |
| 5 | 5.02 | 3.27 | 2.05 | 0.63 | 6 |
| 10 | 10 | 6.15 | 4.4 | 0.72 | 6 |
| 15 | 15.08 | 8.98 | 7.1 | 0.79 | 5.9 |

Concentration 8%

| | | | | | |
|---|---|---|---|---|---|
| 1 | 1.01 | 0.13 | 1.01 | 7.77 | 8.4 |
| 5 | 5.07 | 1.97 | 3.41 | 1.73 | 8 |
| 10 | 10.04 | 3.37 | 7.3 | 2.17 | 7.9 |
| 15 | 15.13 | 0.57 | 15.59 | 27.35 | 7.8 |

TABLE 6
Concentration profile when the anesthetic was maintained at 25° C.:
Concentration profile when the anesthetic was maintained at 25° C.

| | | | | | |
|---|---|---|---|---|---|
| | | | | Ratio | Actual |
| Total Input | Actual Input | Bypass | Tank | (Tank/Bypass) | Conc |

Concentration 2%

| | | | | | |
|---|---|---|---|---|---|
| 1 | 1 | 0.59 | 0.54 | 0.92 | 0.022 |
| 5 | 5.03 | 4.42 | 0.85 | 0.19 | 0.02 |
| 10 | 10 | 9.23 | 1.25 | 0.14 | 0.02 |
| 15 | 15.01 | 14.1 | 1.74 | 0.12 | 0.02 |

Concentration 4%

| | | | | | |
|---|---|---|---|---|---|
| 1 | 1.02 | 0.63 | 0.5 | 0.77 | 0.04 |
| 5 | 5.02 | 4.06 | 1.23 | 0.30 | 0.041 |
| 10 | 9.99 | 8.38 | 2.16 | 0.26 | 0.04 |
| 15 | 15.03 | 12.64 | 3.3 | 0.26 | 0.04 |

Concentration 6%

| | | | | | |
|---|---|---|---|---|---|
| 1 | 1.02 | 0.51 | 0.63 | 1.24 | 0.062 |
| 5 | 5.01 | 3.51 | 1.75 | 0.50 | 0.06 |
| 10 | 10 | 7.13 | 3.44 | 0.48 | 0.06 |
| 15 | 15.04 | 9.85 | 6.2 | 0.63 | 0.058 |

Concentration 8%

| | | | | | |
|---|---|---|---|---|---|
| 1 | 0.99 | 0.5 | 0.62 | 1.24 | 0.081 |
| 5 | 4.99 | 2.62 | 2.69 | 1.03 | 0.079 |
| 10 | 10 | 4.99 | 5.64 | 1.13 | 0.077 |
| 15 | 14.97 | 2.34 | 13.67 | 5.84 | 0.0815 |

As can be appreciated from the above description of the present invention, the anesthetic vaporizer of FIG. 1 can be relatively small. For example, the overall height of the vaporizer can be about 170 mm to about 220 mm and the overall width can be about 1.20 mm to about 140 mm. Further, the vaporizer described herein may be relatively light (e.g., under 6 pounds, such as under 4 pounds). Hence, a vaporizer such as that shown and described herein can be easily transported. The anesthetic vaporizer of the embodiment of FIG. 1 can be

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the described apparatus are merely illustrative; one of ordinary skill in the art could readily determine any number and type of tools, sequences of steps, or other means/options for virtually or actually placing the above-described apparatus, or components thereof, into positions and/or configurations substantially similar to those shown and described herein. Any of the described structures and components could be integrally formed as a single piece or made up of separate sub-components, with either of these formations involving any suitable stock or bespoke components and/or any suitable material or combinations of materials. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. A vaporizer, comprising:
a thermally conductive anesthetic container;
a temperature sensor positioned in the container and in the liquid anesthetic;
a breathing-gas delivery tube having a delivery end positioned in the liquid anesthetic at least about 5 mm beneath the top surface of the liquid anesthetic;
a heater positioned outside the container; and
control circuitry configured to receive signals from the temperature sensor and connected to the heater in order to cause the heater to maintain the temperature of the anesthetic above a predetermined temperature.

2. The vaporizer of claim 1, wherein the delivery end is positioned near a bottom of the container.

3. The vaporizer of claim 1, wherein the temperature sensor is electronic.

4. The vaporizer of claim 1, wherein the temperature sensor is a thermistor.

5. The vaporizer of claim 1, further comprising a breathing-gas conduit for delivering breathing-gas to a manifold.

6. The vaporizer of claim 5, wherein the manifold includes at least one passageway for delivering breathing-gas to the delivery tube.

7. The vaporizer of claim 5, wherein the manifold includes at least one passageway for delivering breathing-gas to a bypass tube.

8. The vaporizer of claim 5, further comprising an anesthetic vapor tube configured to deliver anesthetic vapor from the container to the bypass tube.

9. A method of vaporizing inhalation anesthetic, comprising:
providing a vaporizer having:
a thermally conductive anesthetic container,
a temperature sensor positioned in the container and in the liquid anesthetic,
a breathing-gas delivery tube having a delivery end positioned in the liquid anesthetic,
a heater positioned outside the container, and
control circuitry configured to receive signals from the temperature sensor and connected to the heater to cause the heater to maintain the temperature of the anesthetic above a predetermined temperature;
at least partially filling the container with liquid anesthetic;
providing breathing-gas to the delivery tube to cause breathing-gas to bubble through the liquid anesthetic;
determining the temperature of the liquid anesthetic using the sensor and the control circuitry; and
when the liquid anesthetic temperature is below a set point, causing the heater to provide heat to the container until the temperature achieves the set point.

10. The method of claim 9, further comprising:
providing a breathing-gas manifold;
providing breathing-gas to the manifold; and
adjusting the manifold to provide a portion of the breathing-gas to a bypass tube and another portion of the breathing-gas to the delivery tube.

11. The method of claim 10, further comprising:
providing a vapor tube providing a passageway between the container and the bypass tube; and
delivering anesthetic vapor from the container to the bypass tube via the vapor tube.

12. A vaporizer, comprising:
a thermally conductive anesthetic container containing liquid anesthetic;
a temperature sensor positioned in the container and in the liquid anesthetic;
a breathing-gas delivery tube having a delivery end positioned in the liquid anesthetic beneath the top surface of the liquid anesthetic;
a heater positioned outside the container and configured to supply heat to the liquid anesthetic via conduction through the anesthetic container; and
control circuitry configured to receive signals from the temperature sensor and responsively control the heater to maintain the temperature of the anesthetic at least one of at and above a predetermined temperature.

13. The vaporizer of claim 12, wherein the delivery end of the breathing-gas delivery tube is positioned adjacent, and spaced apart from, an inside bottom of the container.

14. The vaporizer of claim 12, further comprising a breathing-gas conduit for delivering breathing-gas to a manifold.

15. The vaporizer of claim 14, wherein the manifold includes at least one passageway for delivering breathing-gas to the delivery tube.

16. The vaporizer of claim 14, wherein the manifold includes at least one passageway for delivering breathing-gas to a bypass tube.

17. The vaporizer of claim 14, further comprising an anesthetic vapor tube configured to deliver anesthetic vapor from the container to the bypass tube.

18. The vaporizer of claim 14, wherein the manifold includes:
a control mechanism to adjust the amount of anesthetic provided to a patient;
at least one passageway for delivering breathing-gas to the delivery tube;
at least one passageway for delivering breathing-gas to a bypass tube; and
the vaporizer includes an anesthetic vapor tube configured to deliver anesthetic vapor from the container to the bypass tube;
wherein the control mechanism adjustably controls passage of breathing-gas to the delivery and bypass tubes to control relative proportions of anesthetic vapor and breathing-gas in the delivery tube to predetermined levels.
